(19) United States
(12) Patent Application Publication
(10) Pub. No.: US 2019/0125197 A1
(43) Pub. Date: May 2, 2019

(54) Biological Information Detection Apparatus and Biological Information Detection Method

(71) Applicant: Hitachi, Ltd., Tokyo (JP)

(72) Inventors: Nobuhiro FUKUDA, Tokyo (JP); Masashi KIGUCHI, Tokyo (JP); Akiko NAKAJIMA, Tokyo (JP); Takashi NUMATA, Tokyo (JP); Hironori WAKANA, Tokyo (JP); Masuyoshi YAMADA, Tokyo (JP)

(21) Appl. No.: 15/941,471

(22) Filed: Mar. 30, 2018

(30) Foreign Application Priority Data
Oct. 31, 2017 (JP) 2017-210828

(51) Int. Cl. A61B 5/021; G06T 7/90; G06T 7/00; G06T 5/00; A61B 5/024; A61B 5/00

(52) U.S. Cl. CPC A61B 5/02108; G06T 7/90; G06T 7/0012; G06T 5/009; A61B 5/02427; A61B 5/0261; G06T 2207/10024; G06T 2207/30088; G06T 2207/30201; G06T 2207/30104; A61B 5/0077

(57) ABSTRACT

A biological information detection apparatus includes: a camera; a frame image analysis unit that detects a region including pixels having a predetermined skin color, as a skin color region, from a frame image taken using the camera, and detects a signal corresponding to a light wavelength from an image signal of each pixel included in the skin color region, as skin color wavelength data; a skin color wavelength difference detection unit that calculates an average value of differences of the skin color wavelength data from predetermined reference wavelength data for the pixels included in the skin color region, and acquires the average value as average wavelength difference data; a pulse wave signal detection unit that detects a signal obtained by smoothing the average wavelength difference data detected in time series, as a pulse wave signal.

### BACKGROUND OF THE INVENTION

### 1. Field of the Invention

The present invention relates to a biological information detection apparatus and a biological information detection method that detect the dynamic state of a living body in a noncontact manner in real time.

### 2. Description of the Related Art

In recent years, attention has been directed toward the technique of detecting the dynamic state of a living body in a noncontact manner in real time using a microwave or a camera. For example, there is a technique of detecting a heart rate and so forth of a subject from a temporal change in a face image of the subject taken using a camera. With miniaturization of a camera module, this technique has been applied to portable terminals such as smart phones and has been rapidly widespread. Further, this technique has been evolved into a technique of measuring blood pressure of a subject in real time by use of a smart phone or the like.

For example, Patent document 1 discloses the technique of spectrally analyzing RGB time-series signals in a region of interest in a subject's (living body's) image to identify the pulse wave signal originating from the blood vessel in the region of interest. Patent document 2 discloses the technique of detecting a pulse wave signal in each of two sites of a subject from images of the two sites, and finds the pulse wave velocity from the pulse wave signals in the two sites to estimate the blood pressure of the subject. A known example of the method of estimating the blood pressure from the pulse wave velocity is a method using the Moens-Korteweg equation (Refer to non-patent document 1).

### CITATION LIST

### Patent Documents

[Patent document 1] Japanese Laid-open Publication No. 2012-239661

[Patent document 2] Japanese Laid-open Publication No. 2015-54223

### Non-Patent Document

[Non-Patent document 1] Tijsseling A. S., Anderson A., “A. Isebree Moens and D. J. Korteweg: on the speed of propagation of waves in elastic tubes”, BHR Group, Proc. of the 11th Int. Conf. on Pressure Surges (Editor Sandy Anderson), Lisbon, Portugal, October (2012)

According to the inventions described in Patent document 1 and Patent document 2, the pulse wave signal is basically acquired by spectrally analyzing the RGB time-series signals of the pixels in the region of interest in the taken images. The images used for that purpose are taken by detecting RGB light reflected from, for example, a subject's face when the face is illuminated with illuminating light. For this reason, the spectral analysis of the RGB time-series signals means spectral analysis of the time series change in the intensity of three colors RGB in reflected light.

Accordingly, when light is steadily applied onto the subject's face as the region of interest, the pulse wave signal can be stably detected. However, when the intensity of illuminating light or natural light varies irregularly, or the shadow on the subject's face vary due to a motion of the subject, the reflected light from the region of interest also largely varies. For this reason, in some cases, the stable pulse wave signals cannot be acquired from the RGB time-series signals of the reflected light.

In this manner, the conventional techniques of detecting biological information such as the heart rate and blood pressure of the subject from the subject's image are susceptible to an environment such as illumination and natural light, and therefore have a problem that the biological information cannot be stably detected.

### SUMMARY OF THE INVENTION

In consideration of the above-mentioned problem of the conventional technique, an object of the present invention is to provide a biological information detection apparatus and a biological information detection method that can suppress the influence of an environment such as illuminating light and natural light, and stably detect biological information of a subject.

To achieve the object of the present invention, the biological information detection apparatus of the present invention includes a camera that continuously takes images of a subject at a predetermined time interval; a frame image analysis unit that detects a region including pixels having a predetermined skin color, as a skin color region, from a frame image taken using the camera, and detects a signal corresponding to a light wavelength from an image signal of each pixel included in the skin color region, as skin color wavelength data; a skin color wavelength difference detection unit that calculates an average value of differences of the skin color wavelength data detected by the frame image analysis unit from predetermined reference wavelength data or the skin color wavelength data detected in the frame image preceding the current frame image for the pixels included in the skin color region, and detects the average value as average wavelength difference data; and a pulse wave signal detection unit that detects a signal obtained by smoothing the average wavelength difference data detected in time series, as a pulse wave signal.

The present invention provides a biological information detection apparatus and a biological information detection method that can suppress the influence of an environment such as illuminating light and natural light, and stably detect biological information of a subject.

### BRIEF DESCRIPTION OF THE DRAWINGS

FIG. 1 is a block diagram illustrating an example a of a biological information detection apparatus in accordance with a first embodiment;

FIG. 2 is a detailed block diagram illustrating an example of a frame image analysis unit;

FIG. 3 is a view illustrating an example of a space filter used in the frame image analysis unit;

FIG. 4 is a view illustrating an example of an HSV color space and a skin color space;

FIG. 5 is a view illustrating an example of a screen displayed for setting the skin color space;

FIG. 6 is a detailed block diagram illustrating an example of a skin color wavelength difference detection unit;

FIG. 7 is a detailed block diagram illustrating an example of a pulse wave signal detection unit;

FIG. 8 is a block diagram illustrating an example of a biological information detection apparatus in a modification example #1 of the first embodiment;

FIG. 9 is a detailed block diagram illustrating an example of a frame image analysis unit in a modification example #2 of the first embodiment;

FIG. 10 is a detailed block diagram illustrating an example of a skin color wavelength difference detection unit in a modification example #2 of the first embodiment;

FIG. 11 is a block diagram illustrating an example of a biological information detection apparatus in accordance with second embodiment;

FIG. 12 is a detailed block diagram illustrating an example of a local pulse wave detection unit in accordance with the second embodiment;

FIG. 13 is a detailed block diagram illustrating an example of a local pulse wave detection unit in a modification example of the second embodiment;

FIG. 14 is a view illustrating an example of average pulse wave signals each obtained from multiple sub-regions in a frame image and located at the same vertical position, and a basic concept of calculating pulse wave velocity;

FIG. 15 is a view illustrating an example of average pulse wave signals each being an average of sub-regional pulse wave signals from multiple sub-regions in a facial region located at the same vertical position, and a basic concept of calculating pulse wave velocity;

FIG. 16 is a view for describing a method of calculating the pulse wave velocity in the case where some of the laterally-aligned sub-regions located at the same vertical position are pulse wave signal missing sub-regions;

FIG. 17 is a view for describing a method of calculating the pulse wave velocity in the case where all the laterally-aligned sub-regions are pulse wave signal missing sub-regions; and

FIG. 18 is a detailed block diagram illustrating an example of a blood pressure estimation unit in accordance with the second embodiment.

### DETAILED DESCRIPTION OF THE EMBODIMENTS

An embodiment of the present invention will be described below in detail with reference to accompanying figures. Common constituents are given the same reference numerals and description thereof is omitted.

### First Embodiment

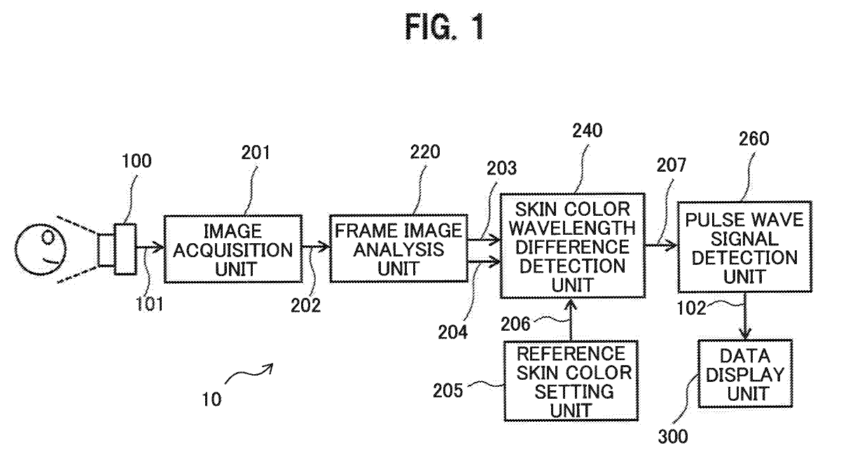

FIG. 1 is a block diagram illustrating an example of a biological information detection apparatus 10 in a first embodiment. As illustrated in FIG. 1, the biological information detection apparatus 10 includes a camera 100, an image acquisition unit 201, a frame image analysis unit 220, a reference skin color setting unit 205, a skin color wavelength difference detection unit 240, a pulse wave signal detection unit 260, and a data display unit 300.

Here, the biological information detection apparatus 10 detects a pulse wave signal of a blood flow flowing in a subject's blood vessel from a change of the subject's skin color over time, which is contained in a subject's image taken using the camera 100, and acquires or estimates heart rate, blood pressure, and so forth. That is, since it is required to detect the change of the subject's skin color over time (time series change), a digital image camera capable of taking a motion image of, for example, about 30 frames per second is used as the camera 100. A subject described herein is a person (human), but may be any animal having a part with less body hair (ex. face) such as a monkey and dog.

Functions of the units constituting the biological information detection apparatus 10 will be described below. In FIG. 1, the image acquisition unit 201 receives an image signal 101 outputted from the camera 100 for each frame, and outputs an. RGB signal 202 for each of the pixels constituting the frame.

The frame image analysis unit 220 receives the RGB signal 202 of each pixel for each frame, which is outputted from the image acquisition unit 201, and outputs a skin color level signal 203 and a skin color wavelength data signal 204 for each pixel. Here, the skin color level signal 203 is a signal indicating that a pixel has a skin color in a predetermined range, and a skin color wavelength data signal 204 is a signal indicating a value of wavelength of the skin color. With reference to FIG. 4, a condition for determining whether the pixel has the skin color in the predetermined range will be described below. A region including pixels having the skin color in the predetermined range is referred to as a skin color region.

The reference skin color setting unit 205 sets a value of a reference skin color wavelength data signal 206 used in the skin color wavelength difference detection unit 240. However, a value of the reference skin color wavelength data signal 206 is set for convenience, and is not limited to any specific value. The value of the reference skin color wavelength data signal 206 may be, for example, “0”.

The skin color wavelength difference detection unit 240 receives the skin color level signal 203, the skin color wavelength data signal 204, and the reference skin color wavelength data signal 206 of each pixel for each frame. Next, for each of sequentially-taken frames, the skin color wavelength difference detection unit 240 finds differences between the skin color level signal 203 of pixels in the skin color region and the reference skin color wavelength data signal 206, and then finds an average value of the differences over the pixels in the skin color region. Then, the found average value is outputted as a time-series skin color wavelength difference data signal 207.

The pulse wave signal detection unit 260 uses the time-series skin color wavelength difference data signal 207 outputted from the skin color wavelength difference detection unit 240 to generate a pulse wave signal 102. Here, the pulse wave signal 102 corresponds to a blood flow rate, blood pressure or the like in the blood vessel, which changes according to subject's heartbeat. That is, in this embodiment, the pulse wave signal 102 of the subject can be detected and further the heart rate of the subject can be detected from the pulse wave signal 102.

The data display unit 300 includes a display device such as an LCD (Liquid Crystal Display), and displays the pulse wave signal 102 and data such as heart rate, which are outputted from the pulse wave signal detection unit 260, on the display device.

The functions of the constituents of the biological information detection apparatus 10 except for the camera 100 and the data display unit 300 cart be achieved by a hardware circuit using, for example, a dedicated integrated circuit (FPGA: Field Programmable Logic Array). Alternatively, the functions can be achieved by a computer provided with a processor, a storage device (semiconductor memory, hard disc device, or the like), and an input/output device (keyboard, mouse, display device or the like) However, in this case, the functions of the constituents of the biological information detection apparatus 10 can be achieved by allowing the processor to execute a predetermined program stored in the storage device.

Subsequently, detailed configuration of the frame image analysis unit 220, the skin color wavelength difference detection unit 240, and the pulse wave signal detection unit 260, which constitute an image processing unit 200, will be described.

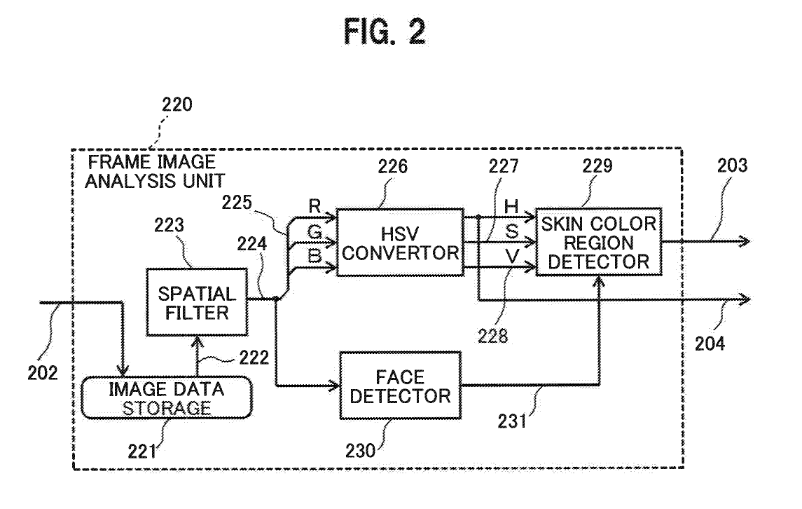

FIG. 2 is a detailed block diagram illustrating an example of the frame image analysis unit 220. As illustrated in FIG. 2, the frame image analysis unit 220 includes an image data storage 221, a spatial filter 223, an HSV convertor 226, a skin color region detector 229, and a face detector 230.

The image data storage 221 receives and holds the RGB signal 202 outputted from the image acquisition unit 201 (See FIG. 1), and outputs a delay REG signal 222 having a line delay by taps of a convolution kernel of the spatial filter 223. The spatial filter 223 receives the delay RBG signal 222, smooths, for example, the. delay RBG signal 222 of a pixel of interest and surrounding pixels by weighted average calculation or the like, and outputs the smoothed signal as a smoothed RGB signal 224.

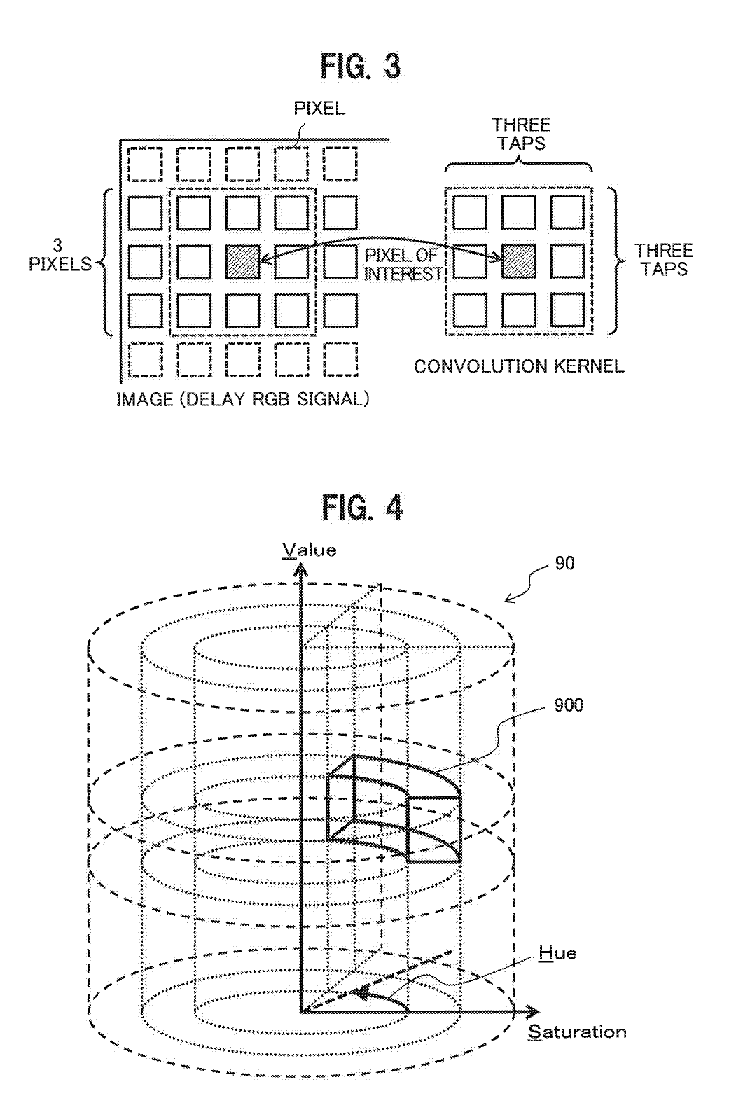

FIG. 3 is a view illustrating an example of the spatial filter 223 used in the frame image analysis unit 220. For example, the spatial filter 223 in this example applies a convolution kernel (determinant) having 3 taps in length and width, that is, 3×3 pixels to smoothing processing of each pixel. In this case, 3×3 pixels about the pixel of interest are subjected to convolution using the convolution kernel, and the acquired value becomes the smoothed RGB signal 224 of the pixel of interest. Components of the determinant of the convolution kernel are, for example, weighted average factors, and may be set as appropriate using average value distribution or Gaussian distribution such that the sum becomes 1.0.

The HSV convertor 226 (See FIG. 2) receives unpack signals 225 of R (red), G (green), and B (blue) which are unpacked from the smoothed RGB signal 224, and converts the unpacked signals 225 into HSV color space signals including a hue signal 204 (H), a saturation signal 227 (S) and a value signal 228 (V). Although differently named, the skin color wavelength data signal 204 outputted from the frame image analysis unit 220 is the same as the hue signal 204 (H) outputted from the HSV convertor 226.

FIG. 4 is a view illustrating an example of an HSV color space 90 and a skin color space 900. As illustrated in FIG. 4, the HSV color space 90 is often represented by cylindrical coordinates. In the cylindrical coordinates, a vertical axis represents value V (Value) indicating color brightness. A radial axis represents saturation S (Saturation) indicating colorfulness. A rotational angle represents hue H (Hue) changing from red to yellow, to green, and finally, purple.

Considering that the hue H is independent from brightness and colorfulness, and the taken image is a detection signal of light, the hue H can be regarded as wavelength of light emitted from each pixel. Thus, in this embodiment, the hue H acquired from each pixel is assumed as a wavelength data signal of the light emitted from each pixel. Similarly, the value V of each pixel can be regarded as the intensity of the light emitted from each pixel.

The skin color space 900 illustrated in FIG. 4 defines a color of human's skin in the HSV color space 90. That is, in this embodiment, it is determined whether or not each of values of the hue signal 204 (H), the saturation signal 227 (S), and the value signal 228 (V) of each pixel outputted from the HSV convertor 226 is included in the skin color space 900. When the values are included in the skin color space 900, the pixel is regarded as a portion of the human's skin.

Thus, the skin color region detector 229 receives the hue signal 204 (H), the saturation signal 227 (S), and the value signal 228 (V) from the HSV convertor 226, and determines whether or not each of the values of the signals is included in the skin color space 900. As a result of the determination, when the value is included in the skin color space 900, “1” is outputted as the skin color level signal 203, and when the value is not included in the skin color space 900, “0” is outputted as the skin color level signal 203.

The human's skin color greatly varies depending on individuals, human race, or how to illuminate. Thus, in this embodiment, the user can set the skin color space 900.

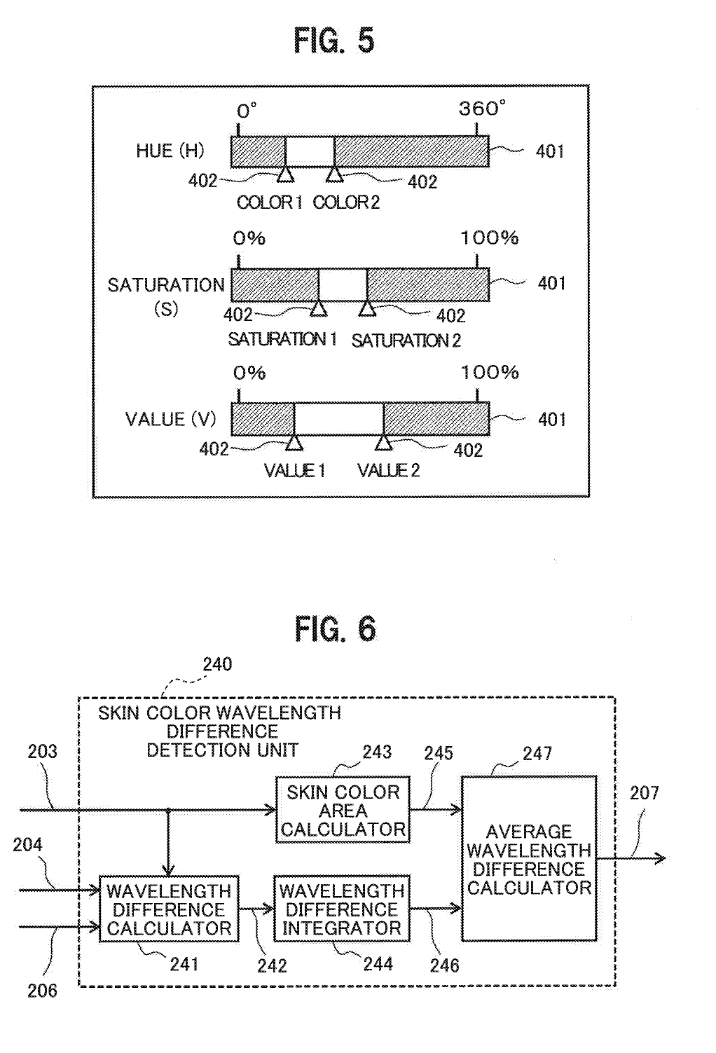

FIG. 5 is a view illustrating an example of a screen displayed for setting the skin color space 900. When setting the skin color space 900 in response to a user's request, the biological information detection apparatus 10 displays a screen as illustrated in FIG. 5 on a predetermined display device. Slide bars 401 indicating the entire ranges of the hue H, the saturation S, and the value V, and two cursors 402 that slide along each of the slide bars 401 are displayed on the screen. The user can freely set the range of the skin color space 900 by appropriately sliding the cursors 402 by use of an input device such as mouse (not illustrated).

For example, FIG. 5 illustrates the slide bar 401 for the hue H in the range of 0 degree to 360 degrees. In this slide bar, 0 degrees (360 degrees) indicate red, 120 degrees indicate green, 240 degrees indicate blue, and the hue H in the skin color space 900 is defined as a region between a color 1 and a color 2. Similarly, for the saturation S, 0% indicates colorless, 100% indicates colorful, and the saturation S in the skin color space 900 is defined as a region between a saturation 1 and a saturation 2. For the value V, 0% indicates dark color, 100% indicates bright color, the value V in the skin color space 900 is defined as a region between a value 1 and a value 2.

In the example illustrated in FIG. 5, the ranges of all of the hue H, the saturation S, and the value V in the skin color space 900 are limited. However, the range of at least the hue H may be limited. In particular, the range of the saturation S may not be limited.

In this manner, the skin color region detector 229 (See FIG. 2) can properly determine whether or not the pixel corresponds to the human's skin portion according to the skin color of the person and the state of illumination. In this specification, a region that is determined as the human's skin portion by the skin color region detector 229, and formed of pixels having the skin color level signal 203 of “1” is referred to as the skin color region.

As described above, the reference skin color setting unit 205 sets the value of the reference skin color wavelength data signal 206, which is used by the skin color wavelength difference detection unit 240. The screen illustrated in FIG. 5 can be used for this setting. The screen in FIG. 5 is used for setting the skin color space 900, but the value of the reference skin color wavelength data signal 206 may be set using the color 1 and the color 2 that defines the range of the hue H. In this case, an intermediate value between the color 1 and the color 2 may be used as the value of the reference skin color wavelength data signal 206.

Referring to FIG. 2, the frame image analysis unit 220 includes the face detector 230. The face detector 230 cut a facial portion from the frame image to be processed by the frame image analysis unit 220. Examples of the method of cutting the facial portion from the frame image include publicly-known Viola-Jones method. In this embodiment, the face detector 230 cuts the facial portion, and outputs a face-detected region signal 231=“1” for each of the pixels of the frame image when the pixel is included in the cut facial portion, and outputs the face-detected region signal 231=“0” for each of the pixels of the frame image when the pixel is not included in the cut facial portion.

For the pixel having the face-detected region signal 231=“0”, the skin color region detector 229 outputs “0” as the skin color level signal 203 without determining whether or not the HSV converted signal of the pixel is included in the skin color space 900 (See FIG. 4). That is, the skin color region detector 229 detects the skin color region only in the face region cut from the overall frame image by the face detector 230.

In this embodiment, the frame image analysis unit 220 has the face detector 230 and however, does not need to have the face detector 230. In this case, the skin color region detector 229 also detects the skin color included in the background of the subject, as the skin color region. However, since the skin color region is not the skin color region that changes according to the subject's heartbeat, the skin color region becomes noise for detecting the pulse wave signal 102 by the biological information detection apparatus 10.

Accordingly, the embodiment in which the frame image analysis unit 220 has the face detector 230 can detect the pulse wave signal 102 more accurately than the embodiment in which the frame image analysis unit 220 does not have the face detector 230.

FIG. 6 is a detailed block diagram illustrating an example of the skin color wavelength difference detection unit 240. As illustrated in FIG. 6, the skin color wavelength difference detection unit 240 includes a wavelength difference calculator 241, a skin color area calculator 243, a wavelength difference integrator 244, and an average wavelength difference calculator 247.

The wavelength difference calculator 241 receives the skin color level signal 203, the skin color wavelength data signal 204, and the reference skin color wavelength data signal 206 of each pixel, and outputs a wavelength difference data signal 242 set as follows according to the value “1” or “0” of the skin color level signal 203 That is, when the skin color level signal 203 is “1”, a value acquired by subtracting the reference skin color wavelength data signal 206 from the skin color wavelength data signal 204 is set as the value of the wavelength difference data signal 242. When the skin color level signal 203 is “0”, “0” is set as the value of the wavelength difference data signal 242. That is, when the target pixel is the pixel included in the skin color region, a difference between the skin color wavelength data signal 204 and the reference skin color wavelength data signal 206 is set as the value of the wavelength difference data signal 242, and when the target pixel is not the pixel included in the skin color region, “0” is set as the value of the wavelength difference data signal 242.

The skin color area calculator 243 receives the skin color level signal 203 indicating that the target pixel is included in the skin color region, counts the number of pixels in the skin color region (skin color level signal 203 is “1”) in the frame image to be processed, and outputs the count value as a skin color area signal 245.

The wavelength difference integrator 244 receives the wavelength difference data signal 242, integrates values of the wavelength difference data signal 242 for all pixels of the frame image, and outputs the integrated value as an integrated wavelength difference data signal 246.

The average wavelength difference calculator 247 receives the skin color area signal 245 and the integrated wavelength difference data signal 246, and outputs a value obtained by dividing the value of the integrated wavelength difference data signal 246 by the value of the skin color area signal 245, as the skin color wavelength difference data signal 207. The skin color wavelength difference data signal 207 means an average value of the wavelength difference data signal 242 for all pixels included in the skin color region of the frame image, that is, a change of the average value of the hue H in the skin color region of the subject from a reference value.

The value of the reference skin color wavelength data signal 206, which is inputted to the skin color wavelength difference detection unit 240, may be “0”. In this case, the skin color wavelength difference data signal 207 outputted from the average wavelength difference calculator 247 is acquired by taking an average of the skin color wavelength data signals 204 over pixels in the skin color region by the number (area) of the skin color region.

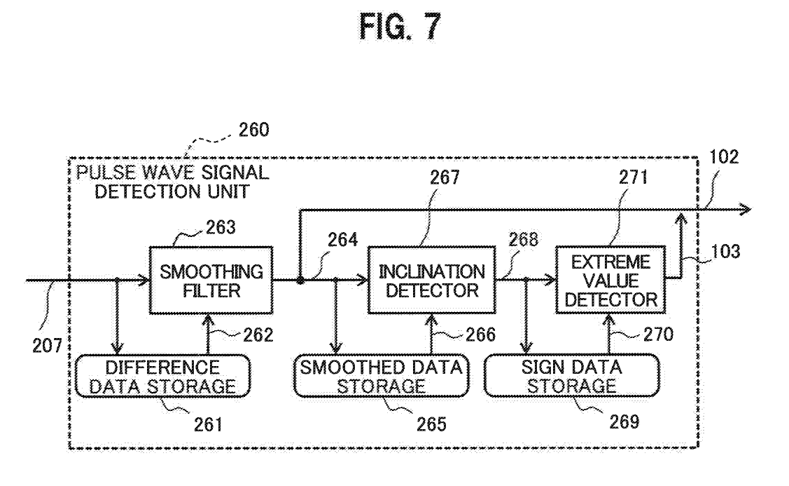

FIG. 7 is a detailed block diagram illustrating an example of the pulse wave signal detection unit 260. As illustrated in FIG. 7, the pulse wave signal detection unit 260 includes a difference data storage 261, a smoothing filter 263, a smoothed data storage 265, an inclination detector 267, a sign data storage 269, and extreme value detector 271. The pulse wave signal detection unit 260 generates the pulse wave signal 102 from the skin color wavelength difference data signal 207 which is outputted from the skin color wavelength difference detection unit 240 for every frame, in other words, outputted from the skin color wavelength difference detection unit 240 over time.

The difference data storage 261 receives and temporarily stores the skin color wavelength difference data signal 207, and outputs a delay skin color wavelength difference data signal 262 that is the skin color wavelength difference data signal 207 for some frames preceding the concerned frame. The smoothing filter 263 receives and smooths the skin color wavelength difference data signal 207 and the delay skin color wavelength difference data signal 262 for some frames, that is, outputs a smoothed wavelength difference data signal 264 obtained by smoothing the skin color wavelength difference data signal 207 for some frames.

The smoothed wavelength difference data signal 264 is a signal obtained by smoothing the change (skin color wavelength difference data signal 207) of the hue H in the skin color region of the subject in terms of time. The time series change of the hue H in the skin color region of the subject can be regarded as corresponding to a change of the blood flow rate in the blood vessel. Thus, the smoothed wavelength difference data signal 264 is outputted to the outside as the pulse wave signal 102 indicating the pulse wave of the blood flow. However, in this embodiment, when the value of the pulse wave signal 102 is a maximum value or a minimum value, a pulse wave extreme value signal 103 indicating the maximum value or the minimum value is added to the pulse wave signal 102.

Thus, the smoothed data storage 265 receives the smoothed wavelength difference data signal 264, stores values for plural frames, and outputs a smoothed delay wavelength difference data signal 266. The smoothed delay wavelength difference data signal 266 is equivalent to the smoothed wavelength difference data signal 264 acquired in frames preceding the frame under processing.

The inclination detector 267 finds a time series change (that is, inclination) of the smoothed wavelength difference data signal 264 from the smoothed delay wavelength difference data signal 266 (that is, the smoothed wavelength difference data signal 264 acquired in frames preceding the concerned frame). Then, a sign of the inclination is outputted as a sign data signal 268.

Specifically, the inclination detector 267 may find the inclination of the smoothed wavelength difference data signal 264 for two continuous frames, or may find the inclination of the smoothed wavelength difference data signal 264 obtained by smoothing on average among multiple continuous frames. In the latter case, the inclination detector 267 may calculate the inclination from an average of the smoothed wavelength difference data signal 264 for multiple continuous frames, and an average of the smoothed wavelength difference data signal 264 for multiple previous continuous frames.

The sign data storage 269 receives the sign data signal 268, stores the values of the sign data signal 268 for multiple frames, and outputs a delay sign data signal 270. The delay sign data signal 270 is equivalent to the sign data signal 268 acquired in frames preceding the frame under processing.

The extreme value detector 271 receives the sign data signal 268 and the delay sign data signal 270 to find a frame having the inclination sign changed from a positive value to a negative value, or a frame having the inclination sign changed from a negative value to a positive value. This means that the smoothed wavelength difference data signal 264 at the time when the found frame is obtained changes from an increase to a decrease or from a decrease to an increase, that is, reaches a maximum value or a minimum value.

Thus, the extreme value detector 271 receives the sign data signal 268 and the delay sign data signal 270, and in the frame having the inclination sign changed from a positive value to a negative value, outputs, for example, “1” as the pulse wave extreme value signal 103. In the frame having the inclination sign changed from a negative value to a positive value, the extreme value detector 271 outputs, for example, “−1” as the pulse wave extreme value signal 103. In the frame having the inclination sign kept unchanged, the extreme value detector 271 outputs, for example, “0” as the pulse wave extreme value signal 103.

As described above, in this embodiment, the smoothing filter 263 smooths the skin color wavelength difference data signal 207 in terms of time, preventing wrong detection of pulse wave due to a minute change of the skin color wavelength difference data signal 207, which is caused by noise and so forth. In this embodiment, the inclination detector 267 detects a change (inclination) of the smoothed wavelength difference data signal 264 for adjacent frames, and based on the change (inclination), the extreme value detector 271 detects a maximum value or a minimum value of difference data. The maximum value or minimum value thus detected is used to count, for example, heart rate.

In the first embodiment described above, the pulse wave signal 102 is generated based on a change of average hue (H) in pixels determined as skin color among the pixels of the taken face image, that is a change of average wavelength of the skin color. In this case, the influences of the value (V) and the saturation (S) on the pulse wave signal 102 are eliminated. For this reason, the influences of natural light and shadows are excluded to provide the technique of detecting the pulse wave signal 102, which is insusceptible to the environment.

In the first embodiment described above, the camera 100 is a visible light color camera, and generates an image signal containing three RGB wavelength components. However, this is merely an example, and the camera 100 may be any camera that can take light reflected from an object (for example, human's face), and output an image signal containing multiple wavelength components For example, at least one of RGB may be included in an infrared or ultraviolet range. To generate such image signal, multiple cameras 100 may be used.

The camera 100 may output an image signal containing two wavelength components. For example, when the image signal outputted from the camera 100 includes only the R signal and the G signal, the generated color space is only the region having the hue (H) in the range of R to G in the HSV color space 90 illustrated in FIG. 4. However, as long as the skin color space 900 is included in the region, the above-described processing can be applied.

In the first embodiment, the RGB signal is converted into the signal of the HSV color space 90. However, the RGB signal may be converted into a signal of another color space including hue and brightness, such as an HSL (Hue, Saturation, Lightness) color space. In any case, an environment-resistant detection method can be provided by detecting a time series change of light wavelength based on the hue signal of the skin color region. In the case of the HSL color space, lightness (L) is acquired as brightness or intensity of light.

### <Modification Example #1 of First Embodiment>

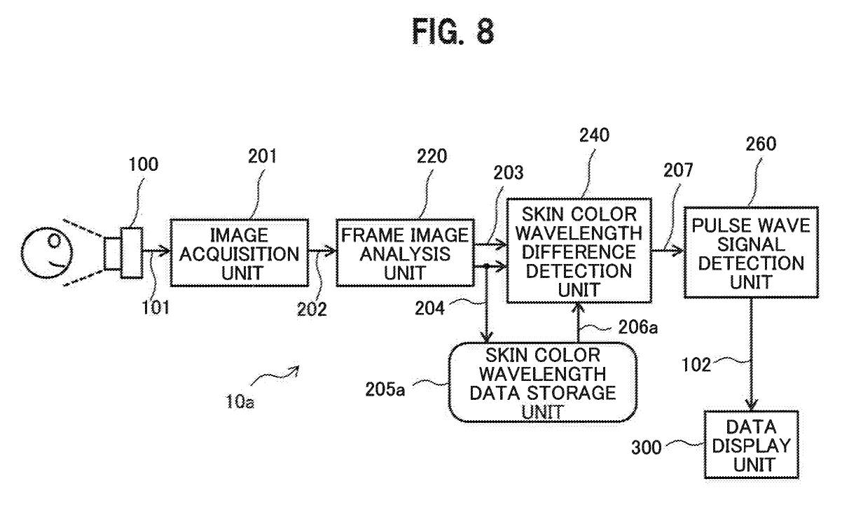

FIG. 8 is a block diagram illustrating an example of a biological information detection apparatus 10 in a modification example #1 of the first embodiment. As illustrated in FIG. 8, the biological information detection apparatus 10a includes the camera 100, the image acquisition unit 201, the frame image analysis unit 220, a skin color wavelength data storage unit 205a, the skin color wavelength difference detection unit 240, the pulse wave signal detection unit 260, and the data display unit 300. The configuration of the biological information detection apparatus 10a is different from the configuration of the biological information detection apparatus 10 in the first embodiment (See FIG. 1) in that the reference skin color setting unit 205 in the biological information detection apparatus 10 is replaced with the skin color wavelength data storage unit 205a.

Here, the functions and detailed configuration of the image acquisition unit 201 and the frame image analysis unit 220 are the same as those in the first embodiment (See FIG. 2 and so on) and thus, description thereof is omitted. In this example, the skin color wavelength data storage unit 205a temporarily stores the skin color wavelength data signal 204 of each pixel outputted from the frame image analysis unit 220 for one or more frames (for example, 3 frames). The skin color wavelength data signal 204 for the frame preceding the current frame by one or more frames is outputted as a delay skin color wavelength data signal 206a.

The functions and detailed configuration of the skin color wavelength difference detection unit 240 and the pulse wave signal detection unit 260 are the same as those in the first embodiment (See FIG. 6, FIG. 7 and so on) and thus, description thereof is omitted. However, this modification example is different from the first embodiment in the signal inputted to the skin color wavelength difference detection unit 240.

In the first embodiment described above, the skin color wavelength difference detection unit 240 (See FIG. 6) receives the skin color level signal 203, the skin color wavelength data signal 204, and the reference skin color wavelength data signal 206 of each pixel. For pixels in the skin color region, which are identified by the skin color level signal 203 for each frame image, the skin color wavelength difference detection unit 240 acquires an average value of the differences between the skin color wavelength data signal 204 and the reference skin color wavelength data signal 206, as the skin color wavelength difference data signal 207.

In contrast, in this modification example, the skin color wavelength difference detection unit 240 receives the skin color level signal 203, skin color wavelength data signal 204, and the delay skin color wavelength data signal 206a of each pixel. For pixels in the skin color region, which are identified by the skin color level signal 203 for each frame image, the skin color wavelength difference detection unit 240 acquires an average value of the differences between the skin color wavelength data signal 204 and the delay skin color wavelength data signal 206a, as the skin color wavelength difference data signal 207. In this modification example, a reference numeral “206” in FIG. 6 denotes the delay skin color wavelength data signal 206a.

Here, the skin color wavelength difference data signal 207 acquired in this modification example can be regarded as a time series change of the average value of the skin color wavelength data signal 204 of the pixels in the skin color region. In contrast, the skin color wavelength difference data signal 207 acquired in the first embodiment is a difference from a reference value (the reference skin color wavelength data signal 206), as well as an average value of the skin color wavelength data signal 204 of pixels in the skin color region. Accordingly, the skin color wavelength difference data signal 207 acquired in this modification example is equivalent to the time-differentiated skin color wavelength difference data signal 207 in the first embodiment.

The pulse wave signal detection unit 260 (See FIG. 7) outputs the signal (smoothed wavelength difference data signal 264) obtained by smoothing the skin color wavelength difference data signal 207 outputted from the skin color wavelength difference detection unit 240 by use of the smoothing filter 263, as the pulse wave signal 102. As described above, the pulse wave signal 102 in the first embodiment indicates a change of the subject's skin color (hue) that changes according to the blood flow rate that increases/decreases with heartbeat. The pulse wave signal 102 is a signal represented by a periodic function having a substantially constant cycle, and heart rate or the like as one of biological information of the subject can be easily acquired from the pulse wave signal 102.

In this modification example, as in the first embodiment, the pulse wave signal 102 is obtained by smoothing the skin color wavelength difference data signal 207 by use of the smoothing filter 263. Accordingly, the pulse wave signal 102 in this modification example is equivalent to a signal acquired by time-differentiating the pulse wave signal 102 in the first embodiment, and is expressed by a periodic function as in the first embodiment. Thus, also in this modification example, the heart rate or the like as one of biological information of the subject can be easily acquired from the pulse wave signal 102 as in the first embodiment.

As described above, since the pulse wave signal 102 acquired in this modification example is acquired based on the skin color wavelength data signal 204 that represents the hue (H) of each pixel in the skin color region, the influences of the value (V) and the saturation (S) on the pulse wave signal 102 are eliminated. For this reason, also in this modification example, the in of natural light and shadows are excluded to provide the technique of detecting the pulse wave signal 102, which is insusceptible to the environment.

### <Modification Example #2 of First Embodiment>

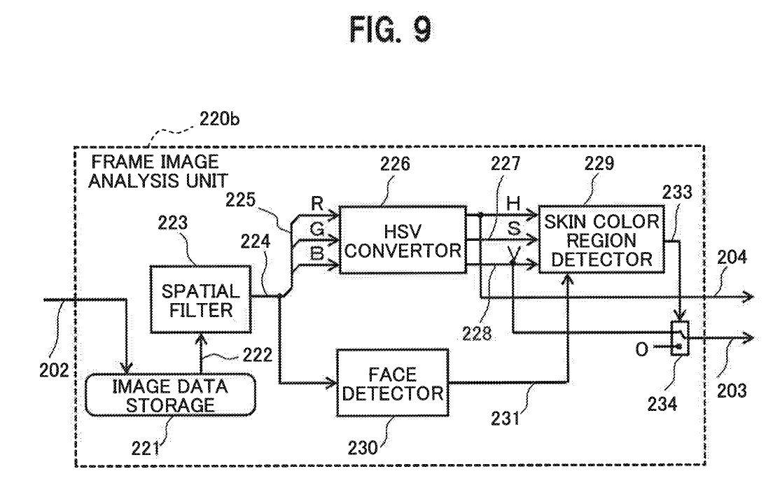

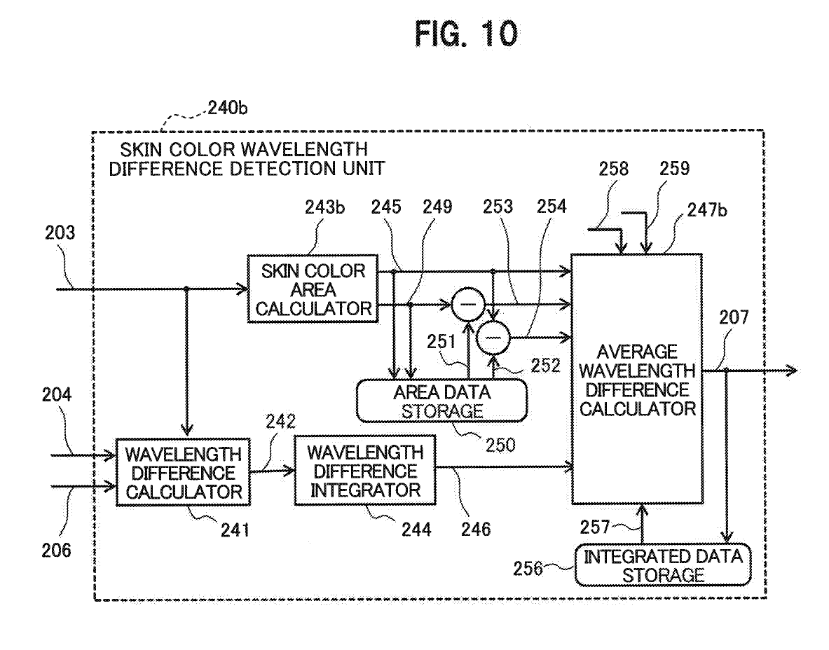

Next, a biological information detection apparatus 10b in a modification example #2 of the first embodiment will be described. The entire configuration of the biological information detection apparatus 10b in this modification example is the same as the configuration of the biological information detection apparatus 10 in the first embodiment in FIG. 1 (See FIG. 1) and thus, illustration thereof is omitted. However, as described with reference to FIG. 9 and FIG. 10, detailed configuration of a frame image analysis unit 220b and a skin color wavelength difference detection unit 240b in this modification example is different from the configuration in the first embodiment.

As described below in detail, the biological information detection apparatus 10b in this modification example is characterized by suppression of lowering of the detection accuracy and wrong detection for the pulse wave signal 102 due to rapid variation in natural light.

FIG. 9 is a detailed block diagram illustrating an example of the frame image analysis unit 220b in the modification example #2 of the first embodiment. As illustrated in FIG. 9, the frame image analysis unit 220b in this modification example includes the image data storage 221, the spatial filter 223, the HSV convertor 226, the skin color region detector 229, the face detector 230, and a signal switch 234. The frame image analysis unit 220b is basically configured by adding the signal switch 234 to the frame image analysis unit 220 in the first embodiment illustrated in FIG. 2. The skin color level signal 203 is outputted from the signal switch 234 rather than the skin color region detector 229.

The functions of the image data storage 221, the spatial filter 223, the HSV convertor 226, and the face detector 230 in this modification example are the same as those in the first embodiment. The function of the skin color region detector 229 is substantially the same as the function in the first embodiment except that an output signal of the skin color region detector 229 is not the skin color level signal 203 (See FIG. 2), but a skin color detection signal 233. However, the skin color level signal 203 in the first embodiment is substantially the same as the skin color detection signal 233 in this modification example.

That is, as in the first embodiment, the skin color region detector 229 in this modification example determines whether or not each of the values of the hue signal 204 (H), the saturation signal 227 (S), and the value signal 228 (V), which are outputted from the HSV convertor 226 is included in the skin color space 900. As a result of this determination, when each value is included in the skin color space 900, “1” is outputted as the skin color detection signal 233, and when each value is not included in the skin color space 900, “0” is outputted as the skin color detection signal 233.

The signal switch 234 receives the value signal 228 (V) from the HSV convertor 226, and the skin color detection signal 233 from the skin color region detector 229. Then, when the value of the skin color detection signal 233 is “1”, the signal switch 234 outputs the value signal 228 (V) from the HSV convertor 226 as the skin color level signal 203b. When the value of the skin color detection signal 233 is “0”, the signal switch 234 outputs “0” as the skin color level signal 203b.

That is, in this modification example, the value of the skin color level signal 203 becomes “0” for pixels outside the skin color region, and becomes the value of the value (V) of the pixel for pixels within the skin color region. The skin color level signal 203 and the skin color wavelength data signal 204 are outputted from the frame image analysis unit 220b.

As in the first embodiment, the face detector 230 cuts a facial portion from the frame image. When the pixel to be processed is included in the cut facial portion, the face detector 230 outputs the face-detected region signal 231=“1”, and when the pixel to be processed is not included in the cut facial portion, the face detector 230 outputs the face-detected region signal 231=“0”. Then, the skin color region detector 229 detects the skin color region only in the facial portion of the frame image, which is cut by the face detector 230.

FIG. 10 is a detailed block diagram illustrating an example of the skin color wavelength difference detection unit 240b in the modification example #2 in the first embodiment. As illustrated in FIG. 10, the skin color wavelength difference detection unit 240b in this modification example includes the wavelength difference calculator 241, a skin color area calculator 243b, an area data storage 250, the wavelength difference integrator 244, an integrated data storage 256, and an average wavelength difference calculator 247b.

Here, the functions of the wavelength difference calculator 241 and the wavelength difference integrator 244 are substantially the same as those in the first embodiment. Accordingly, the wavelength difference calculator 241 receives the skin color level signal 203b, the skin color wavelength data signal 204, and the reference skin color wavelength data signal 206 of each pixel, and outputs the wavelength difference data signal 242 set as follows according to the value of the skin color level signal 203b. That is, when the value of the skin color level signal 203 is “1”, a value acquired by subtracting the reference skin color wavelength data signal 206 from the skin color wavelength data signal 204 is set as the value of the wavelength difference data signal 242. When the value of the skin color level signal 203 is “0”, “0” is set as the value of the wavelength difference data signal 242.

The wavelength difference integrator 244 receives the wavelength difference data signal 242, integrates values of the wavelength difference data signal 242 for all pixels in the concerned frame, and outputs the integrated value as the integrated wavelength difference data signal 246.

In contrast, functions of the skin color area calculator 243b and the average wavelength difference calculator 247b are slightly different from the functions of the skin color area calculator 243 and the average wavelength difference calculator 247 in the first embodiment.

The skin color area calculator 243 receives the skin color level signal 203 representing the value level of the skin color region, counts the number of pixels in the skin color region, that is, the region including no skin color level signal 203 of “0”, for each frame, and outputs the count value as the skin color area signal 245. Further, the skin color area calculator 243 outputs the inputted skin color level signal 203 as a value level signal 249. The area data storage 250 receives and stores the skin color area signal 245 and the value level signal 249, and outputs a delay skin color area signal 252 and a delay value level signal 251.

The integrated data storage 256 temporarily stores values of the skin color wavelength difference data signal 207, which are outputted from the average wavelength difference calculator 247b, for multiple frames, and outputs a delay integrated skin color wavelength data signal 257 that is the skin color wavelength difference data signal 207 for a preceding frame by multiple frames.

The average wavelength difference calculator 247b receives the skin color area signal 245 and the integrated wavelength difference data signal 246, and outputs a value obtained by dividing the value of the integrated wavelength difference data signal 246 by the value of the skin color area signal 245, as the skin color wavelength difference data signal 207. The function of the average wavelength difference calculator 247b is substantially the same as the function of the average wavelength difference calculator 247 in the first embodiment. However, the average wavelength difference calculator 247b in this modification example has following additional functions.

An interframe value level difference signal 253 inputted to the average wavelength difference calculator 247b is a difference between the value level signal 249 for a concerned frame and the value level signal 249 (that is, the delay value level signal 251 read from the area data storage 250) for the frame preceding (for example, immediately preceding) the concerned frame. Accordingly, as the interframe value level difference signal 253 is larger, a change of the value of skin color between frames is larger.

Similarly, an interframe skin color area difference signal 254 inputted to the average wavelength difference calculator 247b is a difference between the skin color area signal 245 for a concerned frame and the skin color area signal 245 (that is, the delay skin color area signal 252 read from the area data storage 250) for a frame preceding (for example, immediately preceding) the concerned frame. Accordingly, as the interframe skin color area difference signal 254 is larger, a change of the area of skin color is larger.

Here, it is assumed that natural light applied to the subject to be processed rapidly changes. In such case, it is considered that the interframe value level difference signal 253 changes larger than the interframe skin color area difference signal 254. In addition, it is considered that the interframe skin color area difference signal 254 rapidly becomes large.

Thus, in this modification example, the average wavelength difference calculator 247b receives the interframe value level difference signal 253, the interframe skin color area difference signal 254, a value level difference threshold 258, and a skin color area difference threshold 259 in addition to the skin color area signal 245 and the integrated wavelength difference data signal 246. Here, the value level difference threshold 258 and the skin color area difference threshold 259 each are a predetermined constant value.

When the interframe value level difference signal 253 is larger than the value level difference threshold 258, the average wavelength difference calculator 247h may output the delay integrated skin color wavelength data signal 257 that is the skin color wavelength difference data signal 207 for the previous frame (for example, immediately preceding frame), as the skin color wavelength difference data signal 207. Alternatively, an average value of the skin color wavelength difference data signal and the delay integrated skin color wavelength data signal 257, which are calculated for the concerned frame, may be outputted as the skin color wavelength difference data signal 207.

Similarly, when the interframe skin color area difference signal 254 is larger than the skin color area difference threshold 259, the average wavelength difference calculator 247b may output the delay integrated skin color wavelength data signal 257 that is the skin color wavelength difference data signal for a previous (for example, immediately preceding) frame, as the skin color wavelength difference data signal 207. Alternatively, an average value of the skin color wavelength difference data signal and the delay integrated skin color wavelength data signal 257, which are calculated for the concerned frame may be outputted as the skin color wavelength difference data signal 207.

In this modification example, when the value and the area in the skin color region rapidly changes due to a rapid change of natural light, a rapid change of the skin color wavelength difference data signal 207 can be suppressed to suppress a rapid change of the pulse wave signal 102. Therefore, in this modification example, even in the case of a rapid change of natural light, lowering the detection accuracy and wrong detection about biological information such as heart rate can be suppressed.

### Second Embodiment

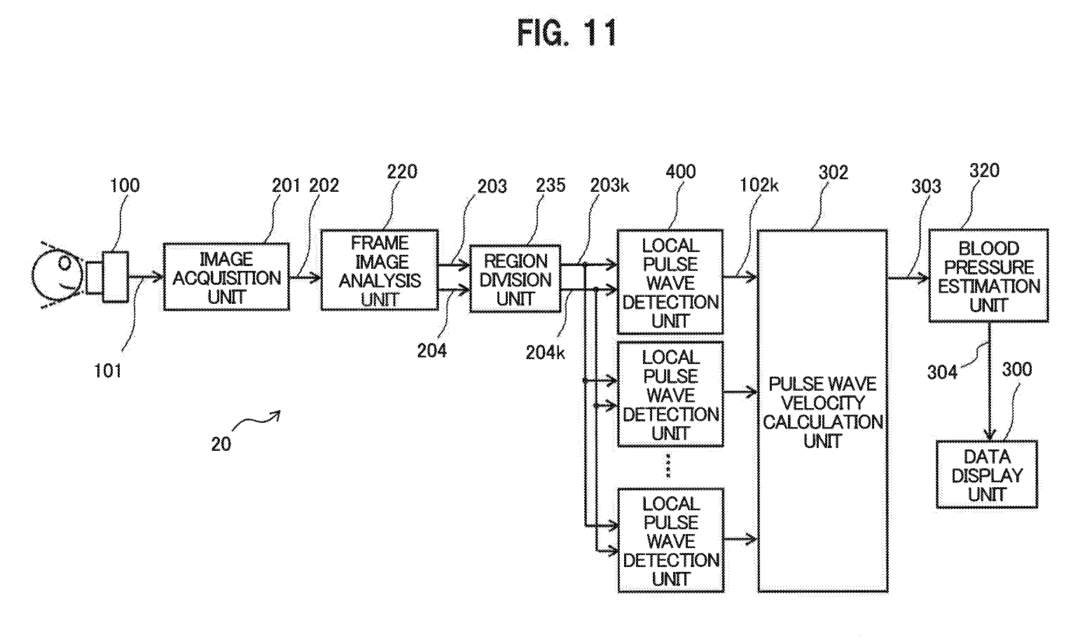

FIG. 11 is a block diagram illustrating an example of a biological information detection apparatus 20 in accordance with a second embodiment. As illustrated in FIG. 11, the biological information detection apparatus 20 includes the camera 100, the image acquisition unit 201, the frame image analysis unit 220, a region division unit 235, multiple local pulse wave detection units 400, a pulse wave velocity calculation unit 302, a blood pressure estimation unit 320, and the data display unit 300. The biological information detection apparatus 20 estimates a blood pressure value of the subject from a time series change of skin color of the subject in an image taken using the camera 100, and displays the estimated blood pressure value on a display device such as LCD via the data display unit 300.

The functions of the constituents of the biological information detection apparatus 20 except for the camera 100 and the data display unit 300 can he achieved by a hardware circuit using, for example, a dedicated integrated circuit (FPGA or the like). Alternatively, the functions can he achieved by a computer provided with a processor, a storage device (semiconductor memory, hard disc device, or the like), and an input/output device (keyboard, mouse, display device or the like). However, in this case, the functions of the constituents of the biological information detection apparatus 20 can be achieved by allowing the processor to execute a predetermined program stored in the storage device.

The functions of the constituents of the biological information detection apparatus 20 will be described in detail. However, the same constituents as the constituents included in the biological information detection apparatus 10 in accordance with the first embodiment are given the same reference numerals and description thereof is omitted.

As in the first embodiment, the camera 100 needs to detect the pulse wave signal based on the time series change of the skin color of the subject, and to estimate blood pressure and therefore, may be a digital video camera capable of taking moving images of about 30 frames per second. Functions and detailed configuration of the image acquisition unit 201 and the frame image analysis unit 220 are the same as those in the first embodiment (See FIG. 2 and so on), and description thereof is omitted.

Accordingly, also in this embodiment, the frame image analysis unit 220 outputs the skin color level signal 203 and the skin color wavelength data signal 204 of each pixel included in the frame image of the target to be processed. Here, the skin color level signal 203 indicates that the image signal of the concerned, pixel is the signal included in the predetermined skin color space 900 (See FIG. 4), that is, in the skin color region. When the pixel is the signal in the skin color region, the skin color wavelength data signal 204 is data corresponding to light wavelength of color expressed by the pixel. However, in this embodiment, the hue signal (H) of the concerned pixel is used as the skin color wavelength data signal 204.

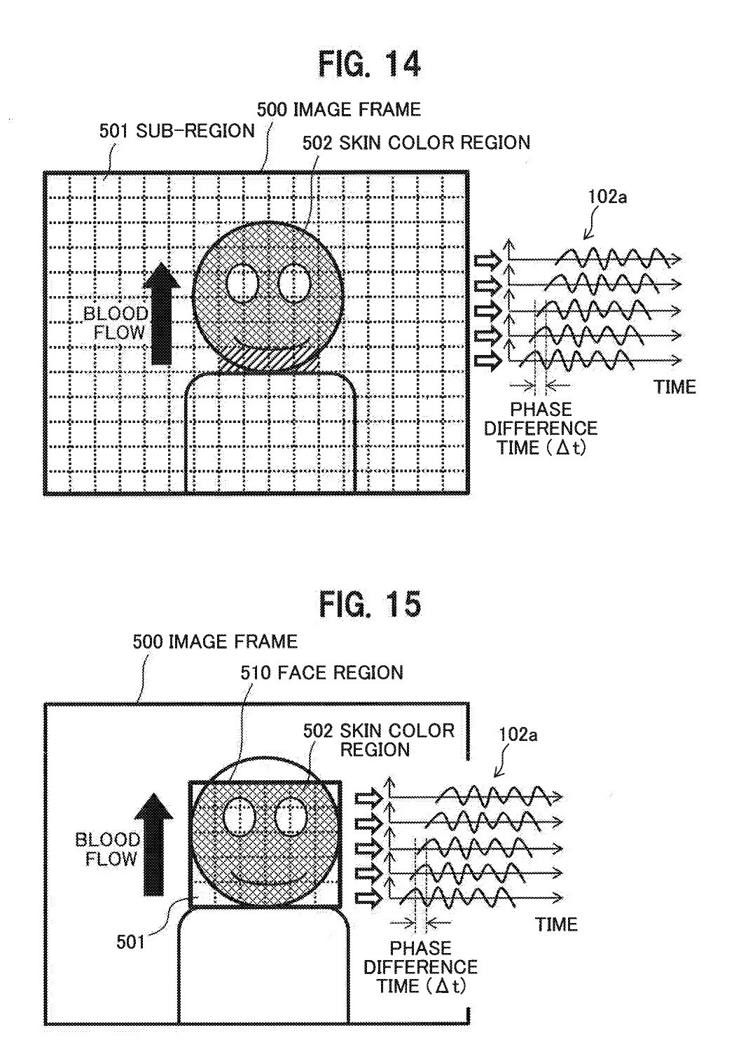

The region division unit 235 divides a frame image to be processed into multiple sub-regions 501 each including, for example, 10×10 pixels (See FIG. 14). The region division unit 235 determines which sub-regions 501 the skin color level signal 203 and the skin color wavelength data signal 204 of each pixel, which are inputted from the frame image analysis unit 220, belong to. The region division unit 235 outputs, as a sub-regional skin color level signal 203k and a sub-regional skin color wavelength data signal 204k for each pixel, signals in which a sub-region number for identifying the sub-region 501 to which the pixel belongs is added to the skin color level signal 203 and the skin color wavelength data signal 204 of the pixel.

The sub-regional skin color level signal 203k and the sub-regional skin color wavelength data signal 204k with the sub-region numbers, which are outputted from the region division unit 235, are classified by the sub-region numbers, and inputted to the local pulse wave detection units 400 assigned for the sub-region numbers. Accordingly, in this embodiment, the same number of local pulse wave detection units 400 as the number of the sub-regions 501 obtained by the region division unit 235 are prepared.

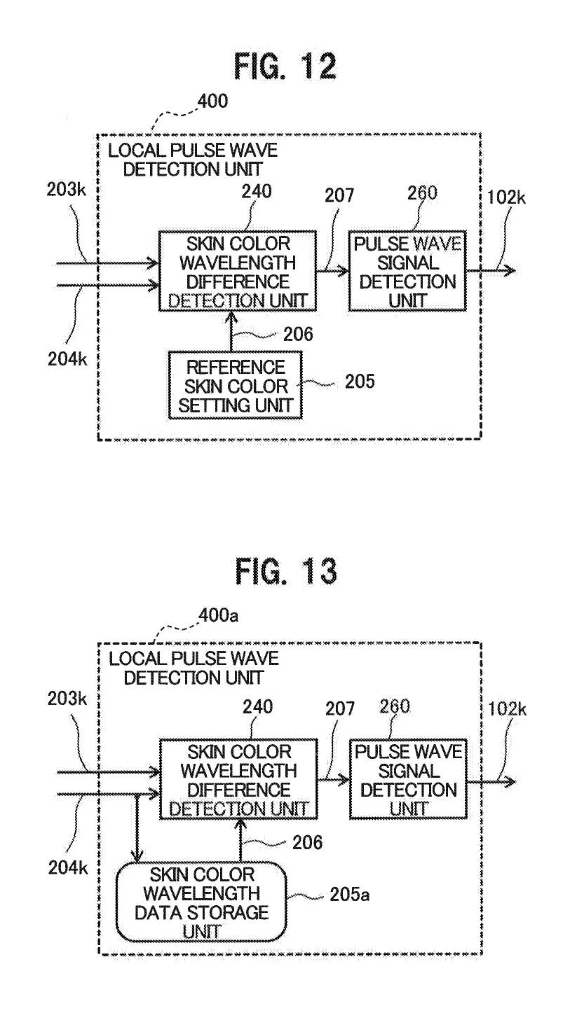

FIG. 12 is a detailed block diagram illustrating an example of the local pulse wave detection unit 400 in accordance with the second embodiment. As illustrated in FIG. 12, the local pulse wave detection unit 400 includes the reference skin color setting unit 205, the skin color wavelength difference detection unit 240, and the pulse wave signal detection unit 260. The local pulse wave detection unit 400 receives the sub-regional skin color level signal 203k and the sub-regional skin color wavelength data signal 204k of the pixel included in the concerned sub-region, and outputs a sub-regional pulse wave signal 102k. Here, the functions and detailed configuration of the reference skin color setting unit 205, skin color wavelength difference detection unit 240, and the pulse wave signal detection unit 260 are the same as those in the first embodiment described with reference to FIG. 6 and FIG. 7, detailed description thereof is omitted.

However, this embodiment is different from the first embodiment in that the skin color wavelength difference detection unit 240 of each local pulse wave detection unit 400 receives only the sub-regional skin color level signal 203k and the sub-regional skin color wavelength data signal 204k of the pixels in its responsible sub-region 501. In summary, the sub-regional pulse wave signal 102k to be outputted from the local pulse wave detection unit 400 is generated for each of the sub-regions 501, by using the sub-regional skin color level signal 203k and the sub-regional skin color wavelength data signal 204k from the pixels in the concerned sub-region 501. That is, in this embodiment, the sub-regional pulse wave signal 102k is not acquired for each frame or facial region, but is acquired for each sub-region 501 with 10×10 pixels, for example, which is a local part of the frame or region.

FIG. 13 is a detailed block diagram illustrating an example of a local pulse wave detection unit 400a in a modification example of the second embodiment. As illustrated in FIG. 13, the local pulse wave detection unit 400a includes a skin color wavelength data storage unit 205a, the skin color wavelength difference detection unit 240, and the pulse wave signal detection unit 260. The local pulse wave detection unit 400a receives the sub-regional skin color level signal 203k and the sub-regional skin color wavelength data signal 204k, and outputs the sub-regional pulse wave signal 102k.

The local pulse wave detection units 400a is configured by replacing the reference skin color setting unit 205 in the local pulse wave detection units 400 illustrated in FIG. 12 with the skin color wavelength data storage unit 205a. That is, the configuration is the same as that in the modification example #1 of the first embodiment. Accordingly, the sub-regional pulse wave signal 102k outputted from the local pulse wave detection unit 400a corresponds to the signal acquired by time-differentiating the sub-regional pulse wave signal 102k outputted from the local pulse wave detection unit 400 in FIG. 12.

Thus, in the second embodiment, the sub-regional pulse wave signal 102k may be outputted from the local pulse wave detection unit 400 illustrated in FIG. 12, or may be outputted from the local pulse wave detection unit 400 illustrated in FIG. 13. Although the second embodiment will be described, below, following description is also applied to the modification example illustrated in FIG. 13.

The pulse wave velocity calculation unit 302 (See FIG. 11) calculates pulse wave velocity based on the sub-regional pulse wave signals 102k outputted from the local pulse wave detection units 400 for the respective sub-regions, and outputs a pulse wave velocity signal 303.

FIG. 14 is a view illustrating an example of average pulse wave signals 102a each obtained from multiple sub-regions 501 in a frame image 500 located at the same vertical position, and the basic concept of calculating the pulse wave velocity. In FIG. 14, the frame image 500 is represented as a rectangle drawn by a thick solid line. Here, the sub-regions 501 are multiple regions into which the frame image 500 is divided and which are drawn by broken lines. An image of a person is displayed in the frame image 500, and a skin color region 502 (shaded portion) is present in the facial portion of the person.

In FIG. 14, the skin color region 502 refers to a region with pixels having the sub-regional skin color level signal 203k of “1”. The sub-regional pulse wave signal 102k is generated using the skin color wavelength difference data signal 207 based on the area (the number of pixels) of the skin color region 502 included in the sub-regions 501 and the sub-regional skin color wavelength data signal 204k of the pixels in the skin color region 502. Accordingly, the sub-regional pulse wave signal 102k cannot be acquired from the sub-regions 501 including no skin color region 502. Further, when the area of the skin color region 502 included in one sub-region 501 is small, the sub-regional pulse wave signal 102k cannot be acquired with high accuracy. Thus, the sub-regional pulse wave signal 102k cannot be generated from the sub-regions 501 when the area ratio of the skin color region 502 to the sub-regions 501 is equal to or smaller than 50%, for example (failure of generation).

Further, as illustrated in FIG. 14, the blood flow in the human's face substantially flows from the lower side to the upper side (in the direction of thick arrow). Accordingly, the sub-regional pulse wave signals 102k having waveforms approximately in phase can be acquired from multiple sub-regions 501 that are located at the same vertical position and aligned in the lateral direction (for example, the hatched sub-regions 501 in FIG. 14) among the sub-regions 501 including the skin color region 502. On the other hand, a phase difference occurs in the waveforms of multiple sub-regional pulse wave signals 102k acquired from the sub-regions 501 located at different vertical positions in the sub-regions 501 including the skin color region 502. The phase difference is a phase difference between the sub-regional pulse wave signals 102k, more specifically, the pulse waves of the blood flow propagating in the blood vessel along with heartbeats.

The average pulse wave signals 102a acquired by averaging the sub-regional pulse wave signals 102k from the sub-regions 501 located at the same vertical position are drawn on the outer right side of the frame image 500 in FIG. 14. A time when the average pulse wave signal 102a reaches an extreme value (a time designated by a frame number or the like) is referred to as an average pulse wave extreme value signal.

Here, the pulse wave velocity (V) can be calculated using a phase difference time Δt between the two average pulse wave signals 102a at the sub-regions 501 located at different vertical positions, and a vertical distance ΔL. That is, the pulse wave velocity (V) is calculated according to an equation: V=ΔL/Δt. The phase difference time Δt between the two average pulse wave signals 102a can be readily found as a time difference between average pulse wave extreme value signals 103a of the two average pulse wave signals 102a.

The average pulse wave signal 102a is preferably an average of all the sub-regional pulse wave signals 102k acquired from the sub-regions 501 located at the corresponding vertical position, but may be the sub-regional pulse wave signal 102k acquired from one of the sub-regions 501 located at the corresponding vertical position. However, generally, the use of the average of measurement values can achieve higher accuracy.

FIG. 15 is a view illustrating an example of the average pulse wave signals 102a each being an average of the sub-regional pulse wave signals 102k obtained from multiple sub-regions 501 in a facial region 510 located at the same vertical position, and the basic concept of calculating the pulse wave velocity. In FIG. 15, the facial region 510 detected by the face detector 230 is displayed as a rectangle drawn by a thick solid line, and the facial region 510 are divided into multiple sub-regions 501 by broken lines. Further, the skin color region 502 (shaded portion) is present in the facial region 510.

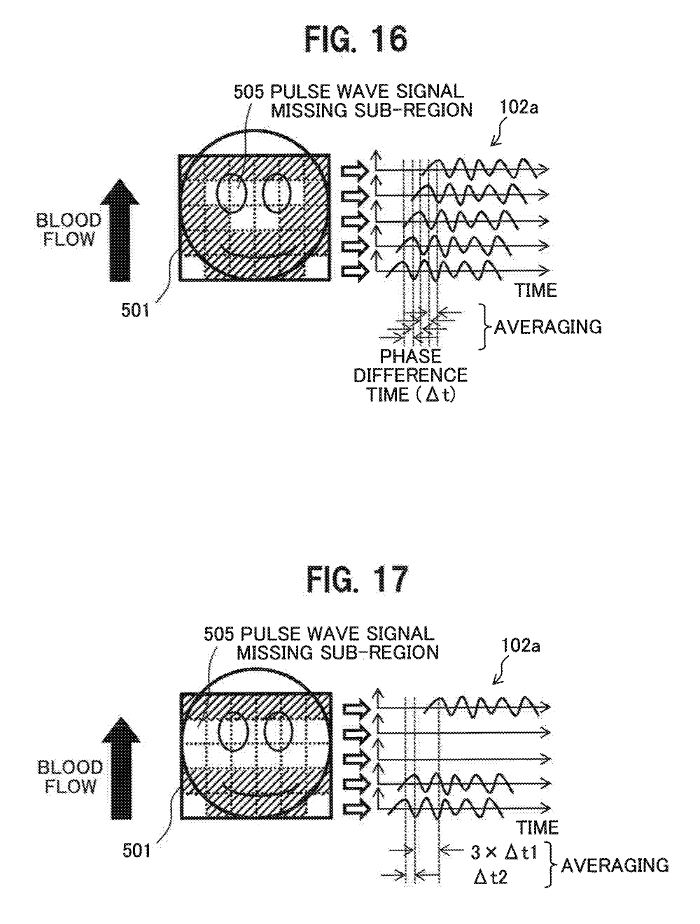

FIG. 15 is different from FIG. 16 in that the sub-regions 501 for finding the sub-regional pulse wave signals 102k are not set in the entire frame image 500, but set in the facial region 510 detected by the face detector 230. Except this, FIG. 15 is the same as FIG. 14 and description thereof is omitted.

FIG. 16 is a view for describing a method of calculating the pulse wave velocity in the case where some of the laterally-aligned sub-regions 501 located at the same vertical position are pulse wave signal missing sub-regions 505. Here, the pulse wave signal missing sub-region 505 refers to a sub-region 501 from which the sub-regional pulse wave signals 102k cannot be acquired, and in FIG. 16, is represented as a hollow sub-region 501. The hatched sub-regions 501 in FIG. 16 represent the sub-regions 501 from which the sub-regional pulse wave signals 102k are acquired.

As described above, to calculate pulse wave velocity, first, an average of the sub-regional pulse wave signals 102k, which are acquired from multiple sub-regions 501 located at the same vertical position and different lateral positions, that is, the average pulse wave signal 102a is calculated. In FIG. 16, the laterally-aligned sub-regions 501 include pulse wave signal missing sub-regions 505 in some part, but also include sub-regions 501 from which the sub-regional pulse wave signals 102k are acquired. In such case, the average pulse wave signal 102a can be acquired by averaging the sub-regional pulse wave signals 102k of the sub-regions 501 from which the sub-regional pulse wave signals 102k are acquired.

To put it more specifically using the example in FIG. 16, the laterally-aligned sub-regions 501 located at the second vertical position from the top include six sub-regions 501, four of which are pulse wave signal missing sub-regions 505, and two of which are sub-regions where the sub-regional pulse wave signals 102k are acquired. In such case, the average pulse wave signal 102a at the vertical position can be acquired by averaging the sub-regional pulse wave signals 102k from the two sub-regions 501.

When the average pulse wave signal 102a is acquired at each vertical position in this manner, the average pulse wave extreme value signal can be acquired from each of the average pulse wave signals 102a. Then, an average value Ave (Δt) can be found as an average of the phase difference time Δt between the average pulse wave extreme value signals at adjacent vertical positions. Here, the pulse wave velocity (V) can be found according to an equation: V=ΔL/Ave (Δt).

FIG. 17 is a view for describing a method of calculating the pulse wave velocity in the case where all the laterally-aligned sub-regions 501 are pulse wave signal missing sub-regions 505. In the example illustrated in FIG. 17, at the second and third vertical positions from the top, all the laterally-aligned sub-regions 501 are the pulse wave signal missing sub-regions 505. Thus, at these vertical positions, the average pulse wave signals 102a cannot be acquired. However, at the first, fourth, and fifth vertical positions, average pulse wave signals 102a are acquired.

In such a case, a phase difference time Δt1 per vertical distance corresponding to one sub-region is found from the average pulse wave signals 102a at the first and fourth vertical positions from the top, and a phase difference time Δt2 is found from the average pulse wave signals 102a at the fourth and fifth vertical positions from the top. Given that an average value of the phase difference times Δt1 and Δt2 is expressed as Ave (Δt1, Δt2), the pulse wave velocity (V) can be found according to an equation: V=ΔL/Ave (Δt1, Δt2).

As described above, even when all the laterally-aligned sub-regions 501 are the pulse wave signal missing sub-regions 505 at any vertical position, as long as the average pulse wave signals 102a are acquired at vertical positions above and below the vertical position, the phase difference time Δt per vertical distance corresponding to one sub-region can be found using the average pulse wave signals 102a. Accordingly, the pulse wave velocity (V) can be found.

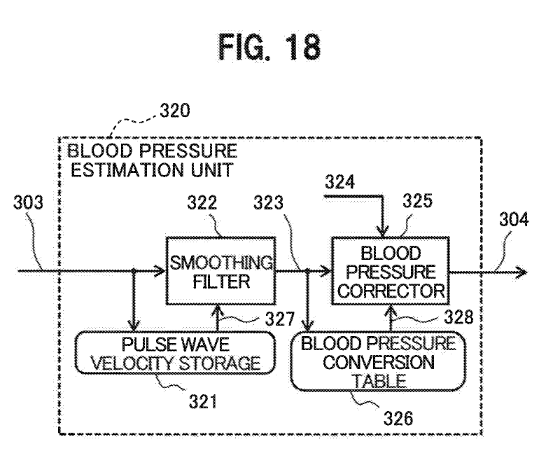

FIG. 18 is a detailed block diagram illustrating an example of the blood pressure estimation unit 320 in accordance with the second embodiment. As illustrated in FIG. 18, the blood pressure estimation unit 320 includes a pulse wave velocity storage 321, a smoothing filter 322, a blood pressure conversion table 326 and a blood pressure corrector 325.

Here, the pulse wave velocity storage 321 stores values of the pulse wave velocity signal 303 inputted over the multiple frames, and outputs a delay pulse wave velocity signal 327. The smoothing filter 322 averages the pulse wave velocity signals 303 and the delay pulse wave velocity signals 327 inputted over the multiple frames, and outputs a smoothed pulse wave velocity signal 323.

The blood pressure conversion table 326 receives the smoothed pulse wave velocity signal 323, searches the table, and outputs a blood pressure conversion signal 328 on which blood pressure is based. According to the Moens-Horteweg equation, a blood pressure value (P) in the diastolic phase is proportional to the square of the pulse wave velocity (PWV). That is, an equation: P=c×PWV² is satisfied. However, a proportionality constant c depends on various kinds of biological information (age, sex, blood vessel radius, blood density, and so forth) of the subject. Thus, the blood pressure conversion table 326 receives the value of the smoothed pulse wave velocity signal 323 as the pulse wave velocity (PWV), and outputs the blood pressure value for predetermined typical biological information as the blood pressure conversion signal 328.

The blood pressure corrector 325 receives the smoothed pulse wave velocity signal 323, the blood pressure conversion signal 328, and a blood pressure correction parameter 324, corrects the blood pressure conversion signal 328, and outputs an estimated blood pressure value 304. Here, the blood pressure correction parameter 324 is a numerical value necessary for determining the proportionality constant c, such as age, sex, blood vessel radius, and blood density. That is, the blood pressure corrector 325 corrects the blood pressure value for typical biological information acquired from the blood pressure conversion table 326 according to biological information of the subject.

In this embodiment, the blood pressure estimation unit 320 estimates the blood pressure value of the subject by using the pulse wave velocity signal 303, the blood pressure conversion table 326, and the blood pressure correction parameter 324 and however, the estimated blood pressure value 304 of the subject may be calculated according to a mathematical model such as the Moens-Korteweg equation.

In the second embodiment, the multiple sub-regional pulse wave signals 102k acquired from the multiple sub-regions 501 including the skin color region 502 is generated based on the sub-regional skin color wavelength data signal 204k corresponding to the hue (H) acquired from pixels in the skin color region 502. In this case, the influences of the value (V) and the saturation (S) on the sub-regional pulse wave signals 102k are eliminated. In summary, in the second embodiment, the estimated blood pressure value 304 is calculated using the multiple sub-regional pulse wave signals 102k, with the influences of the value (V) and the saturation (S) being eliminated. Accordingly, in the second embodiment, the estimated blood pressure value 304, with the influence of natural light, that is, the influences of the value (V) and the saturation (S) being eliminated, can be acquired.

The present invention is not limited to the above-mentioned embodiments and modification examples, and includes other various modification examples. For example, the above-mentioned embodiments and modification examples describe the present invention in detail to facilitate understanding of the present invention, and do not necessarily include all the described constituents. In addition, a unit of the configuration of any embodiment or modification example may be replaced with the configuration of another embodiment or modification example. Alternatively, the configuration of any embodiment or modification example may be combined with the configuration of another embodiment or modification example o Further, part of the configuration of each of the embodiments and modification examples may be altered by addition, deletion, or replacement of a configuration in another embodiment or modification example.

What is claimed is:

1. A biological information detection apparatus comprising:
a camera that continuously takes images of a subject at a predetermined time interval;
a frame image analysis unit that detects a region including pixels having a predetermined skin color, as a skin color region, from a frame image taken using the camera, and detects a signal corresponding to a light wavelength from an image signal of each pixel included in the skin color region, as skin color wavelength data;
a skin color wavelength difference detection unit that calculates an average value of differences of the skin color wavelength data detected by the frame image analysis unit from predetermined reference wavelength data or the skin color wavelength data detected in the frame image preceding the current frame image over the pixels included in the skin color region, and detects the average value as average wavelength difference data; and
a pulse wave signal detection unit that detects a signal obtained by smoothing the average wavelength difference data detected in time series, as a pulse wave signal.

2. The biological information detection apparatus according to claim 1, wherein
the frame image analysis unit converts the image signal of each pixel in the frame image taken using the camera into a color space signal including at least hue and brightness, regards a value of the hue in the color space signal as the light wavelength, and determines whether color of the pixel is the predetermined skin color based on at least the value of the hue.

3. The biological information detection apparatus according to claim 2, wherein
the color space is a hue, saturation, value (HSV) color space having independent axes of hue H, saturation S, and value V.

4. The biological information detection apparatus according to claim 2, wherein
the color space is a hue, saturation, lightness (HSL) color space having independent axes of hue H, saturation. S, and lightness L.

5. The biological information detection apparatus according to claim 1, further comprising a facial region detection unit that detects a facial region of the subject from the frame image, wherein
the frame image analysis unit detects the skin color region only in the facial region detected by the facial region detection unit.

6. The biological information detection apparatus according to claim 1, further comprising:
a region division unit that divides the frame image into multiple sub-regions, and classifies the skin color wavelength data of the pixels in the skin color region detected b the frame image analysis unit for each of the sub-regions;
local pulse wave detection unit that detects the average wavelength difference data for each of the sub-regions where the skin color region is detected, based on the skin color wavelength data of the pixels in the skin color region classified for the sub-region, and detects a local pulse wave signal in each of the sub-regions from the average wavelength difference data detected in time series for the sub-region; and
a blood pressure estimation unit that estimates blood pressure of the subject from multiple local pulse wave signals detected in at least two sub-regions located at different positions along a blood flow of the subject, among a plurality of the local pulse wave signals detected by the local pulse wave detection unit.

7. The biological information detection apparatus according to claim 6, wherein
the blood pressure estimation unit includes a pulse wave velocity calculator that calculates a pulse wave velocity that is a velocity at which the pulse wave signal propagates, by using phase difference time between the multiple local pulse wave signals detected in the at least two sub-regions located at the different positions along the blood flow of the subject, and a clearance along the blood flow between the sub-regions from which the pulse wave signals are detected, and
the blood pressure estimation unit estimates blood pressure of the subject by using the pulse wave velocity calculated by the pulse wave velocity calculator.

8. A biological information detection method, wherein a biological information detection apparatus that includes a camera, and detects biological information of a subject, based on frame images of the subject continuously taken using the camera at a predetermined time interval executes:
a frame image analysis step of detecting a region including pixels having a predetermined skin color, as a skin color region, from a frame image taken using the camera, and detecting a signal corresponding to a light wavelength from an image signal of each pixel included in the skin color region, as skin color wavelength data;
a skin color wavelength change detection step of calculating an average value of differences of the skin color wavelength data detected in the frame image analysis step from predetermined reference wavelength data or the skin color wavelength data detected in the frame image preceding the current frame image over the pixels included in the skin color region, and detecting the average value as average wavelength difference data; and
a pulse wave signal detection step of detecting a signal obtained by smoothing the average wavelength difference data detected in time series, as a pulse wave signal.

9. The biological information detection method according to claim 8, wherein
in the frame image analysis step, the biological information detection apparatus converts the image signal of each pixel in the frame image taken using the camera into a color space signal including at least hue and brightness, regards a value of the hue in the color space signal as the light wavelength, and determines whether color of the pixel is the predetermined skin color based on at least the value of the hue.

10. The biological information detection method according to claim 9, wherein
the color space is an HSV color space having independent axes of hue H, saturation S, and value V.

11. The biological information detection method according to claim 9, wherein
the color space is an HSL color space having independent axes of hue H, saturation S, and lightness L.

12. The biological information detection method according to claim 9, wherein
the biological information detection apparatus further executes a facial region detection step of detecting a facial region of the subject from the frame image, and
in the frame image analysis step, the biological information detection apparatus detects the skin color region only in the facial region detected in the facial region detection step.

13. The biological information detection method according to claim 8, wherein
the biological information detection apparatus further executes:
a region division step of dividing the frame image into multiple sub-regions, and classifying the skin color wavelength data of the pixels in the skin color region detected in the frame image analysis step for each of the sub-regions;
a local pulse wave detection step of detecting the average wavelength difference data for each of the sub-regions where the skin color region is detected, based on the skin color wavelength data of the pixels in the skin color region classified for the sub-region, and detecting a local pulse wave signal in each of the sub-regions from the average wavelength difference data detected in time series for the sub-region; and
a blood pressure estimation step of estimating blood pressure of the subject from multiple local pulse wave signals detected in at least two sub-regions located at different positions along a blood flow of the subject, among a plurality of the local pulse wave signals detected in the local pulse wave detection step.

14. The biological information detection method according to claim 13, wherein
the biological information detection apparatus executes a pulse wave velocity calculation step of calculating a pulse wave velocity that is a velocity at which the pulse wave signal propagates, by using phase difference time between the at least two local pulse wave signals detected in the at least two sub-regions located at the different positions along the blood flow of the subject, and a clearance along the blood flow between the sub-regions from which the pulse wave signals are detected, and
in the blood pressure estimation step, the biological information detection apparatus estimates blood pressure of the subject by using the calculated pulse wave velocity.
